(19) United States
(12) Patent Application Publication
(10) Pub. No.: US 2017/0188853 A1
(43) Pub. Date: Jul. 6, 2017

(54) IMAGING APPARATUS, IMAGING METHOD AND MEDICAL IMAGING SYSTEM

(71) Applicant: Sony Corporation, Tokyo (JP)

(72) Inventors: Isamu Nakao, Tokyo (JP); Eiichi Tanaka, Chiba (JP); Takuya Kishimoto, Tokyo (JP)

(73) Assignee: Sony Corporation, Tokyo (JP)

(21) Appl. No.: 15/313,573

(22) PCT Filed: May 20, 2015
(86) PCT No.: PCT/JP2015/002521

§ 371 Date: Nov. 23, 2016

(30) Foreign Application Priority Data
May 30, 2014 (JP) 2014-112436
Dec. 24, 2014 (JP) 2014-260054

(51) Int. Cl. A61B 5/026; A61B 1/00; A61B 5/00; A61B 1/04

(52) U.S. Cl. CPC A61B 5/0261; A61B 1/04; A61B 1/00045; A61B 5/742

(57) ABSTRACT

Some embodiments relate to an imaging apparatus. The imaging apparatus includes a light source that emits coherent light to image an object. The imaging apparatus also includes an optical apparatus that optically processes light from the object. The optical apparatus has a numerical aperture. The imaging apparatus also includes an imager that receives the light optically processed by the optical apparatus. The imaging apparatus also includes circuitry configured to obtain speckle data based on the light received by the imager and control the numerical aperture of the optical apparatus based on the speckle data.

### CROSS REFERENCE TO RELATED APPLICATIONS

This application claims the benefit of Japanese Priority Patent Application JP 2014-112436 filed May 30, 2014, and Japanese Priority Patent Application JP 2014-260054 filed Dec. 24, 2014, the entire contents of which are incorporated herein by reference.

### TECHNICAL FIELD

The present technology is related to a fluid analysis device, a fluid analysis method, a program and a fluid analysis system, and specifically, is related to a technology of detecting and analyzing fluid such as a blood flow.

### BACKGROUND ART

In a medical field, for example, at the time of medical treatment, the flow of fluid such as blood or the like has to be detected in some cases. In PTL 1, a technology of an imaging system that captures a coherent light image of a blood vessel area is disclosed. According to this technology, for example, an internal blood vessel of an organ and blood that covers a living body surface can be distinguished.

### CITATION LIST

### Patent Literature

PTL 1: Japanese Unexamined Patent Application Publication No. 2009-136396

### SUMMARY

### Technical Problem

However, there has been a demand of enhancing the detection sensitivity of the fluid such as the blood flow in the imaging system in the related art described above.

Therefore, a main object of the disclosure is to provide a fluid analysis device, a fluid analysis method, a program, and a fluid analysis system that can enhance the detection accuracy of a flow of fluid.

### Solution to Problem

Some embodiments relate to an imaging apparatus that includes: a light source that emits coherent light to image an object;

an optical apparatus that optically processes light from the object, the optical apparatus having a numerical aperture; an imager that receives the light optically processed by the optical apparatus; and circuitry configured to obtain speckle data based on the light received by the imager and control the numerical aperture of the optical apparatus based on the speckle data.

Some embodiments relate to an imaging method, including: emitting coherent light to image an object; optically processing light from the object using an optical apparatus having a numerical aperture; obtaining speckle data based on the light optically processed by the optical apparatus; and

controlling the numerical aperture of the optical apparatus based on the speckle data.

Some embodiments relate to a medical imaging system, including:

an imaging apparatus, including: a light source that emits coherent light to image an object; an optical apparatus that optically processes light from the object, the optical apparatus having a numerical aperture; an imager that receives the light optically processed by the optical apparatus; and

circuitry configured to obtain speckle data based on the light received by the imager and control the numerical aperture of the optical apparatus based on the speckle data.

### Advantageous Effects of Invention

According to the present disclosure, the detection accuracy of the flow of the fluid can be enhanced. Further, the effects described herein are examples, are not intended to limit the disclosure, and may be any one of effects described in the disclosure.

### BRIEF DESCRIPTION OF DRAWINGS

FIG. 1 is a diagram schematically illustrating a configuration example of a fluid analysis device 1 of a first embodiment of the disclosure.

FIG. 2 is a diagram schematically illustrating a configuration example of another fluid analysis device 1 of the first embodiment.

FIG. 3 is a diagram illustrating a state of forming an image with light using an image forming optical system 3.

FIG. 4A is a diagram illustrating an example of a speckle image.

FIG. 4B is a diagram illustrating an example of a speckle image.

FIG. 5 is a diagram illustrating a state of mapping the speckle image.

FIG. 6 is a graph illustrating a relationship between a numerical aperture and speckle contrast.

FIG. 7 is a graph illustrating a relationship between the numerical aperture and a difference between the speckle contrast of the blood flow X and speckle contrast other than that of the blood flow X.

FIG. 8 is a diagram schematically illustrating a configuration example of a fluid analysis device 11 of the modification example of the first embodiment.

FIG. 9A is a diagram illustrating a configuration of a tool 100 that circulates the fluid.

FIG. 9B is a diagram illustrating a configuration of a tool 100 that circulates the fluid.

FIG. 9C is a diagram illustrating a configuration of a tool 100 that circulates the fluid.

FIG. 10A is a diagram illustrating an example of the speckle image.

FIG. 10B is a diagram illustrating an example of the speckle image.

FIG. 10C is a diagram illustrating an example of the speckle image.

FIG. 10D is a diagram illustrating an example of the speckle image.

FIG. 11 is a graph illustrating a relationship between the numerical aperture and the speckle contrast with respect to the speckle images illustrated in FIGS. 10A to 10D.

FIG. 12 is a graph illustrating a relationship between the numerical aperture and the difference between the speckle contrast of the blood flow X and the speckle contrast other than that of the blood flow X with respect to the speckle images illustrated in FIGS. 10A to 10D.

FIG. 13 shows a diagram of a medical imaging system, specifically, an endoscopy system, according to some embodiments.

### DESCRIPTION OF EMBODIMENTS

Hereinafter, embodiments of the disclosure are described in detail with reference to the accompanying drawings. Further, the disclosure is not limited to the embodiments described below. In addition, the descriptions are presented as follows.

1. First embodiment

(Example of fluid analysis device for adjusting numerical aperture based on speckle data)

2. Second embodiment

(Example of fluid analysis system adjusting numerical aperture based on speckle data)

### 1. First Embodiment

<Configuration of Fluid Analysis Device 1>

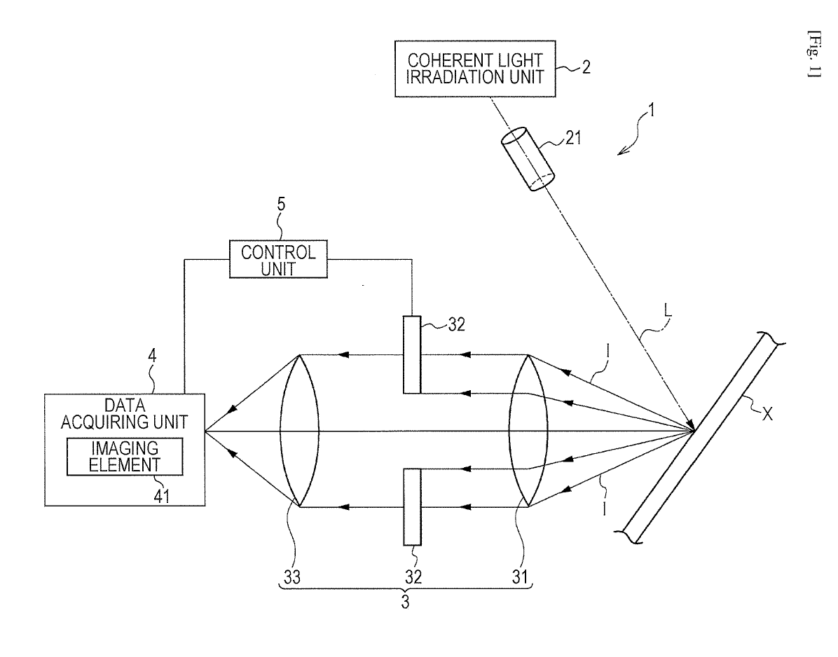

First, a fluid analysis device 1 of the first embodiment of the disclosure is described. FIG. 1 is a diagram schematically illustrating the fluid analysis device 1 of the embodiment. The fluid analysis device 1 of the embodiment includes a coherent light irradiation unit 2 that irradiates the fluid X with coherent light L, an image forming optical system 3 that forms an image with light applied to the fluid X, and a data acquiring unit 4 that acquires the speckle data of the fluid, and the image forming optical system 3 adjusts the numerical aperture based on the speckle data.

In addition, in the fluid analysis device 1 of the embodiment, the fluid X is not particularly limited as long as it has the light scattering material. However, it is preferable that blood is used. At this point, the fluid analysis device 1 functions as a blood flow analysis device, so that while performing medical treatment on an animal such as a human, the fluid analysis device 1 can detect the blood flow thereof and analyze the blood flow. Hereinafter, the fluid X is generally described as the blood flow X.

<Coherent Light Irradiation Unit 2>

The coherent light irradiation unit 2 included in the fluid analysis device 1 of the embodiment irradiates the blood flow X with the coherent light L. The coherent light irradiation unit 2 can be used as a light source for irradiation with the coherent light L. As described below, the coherent light L is not particularly limited as long as the speckle data can be obtained from light applied to the blood flow X, and a laser beam can be used as the coherent light L. In addition, a beam expander 21 can be provided between the coherent light irradiation unit 2 and the blood flow X.

<Image Forming Optical System 3>

The image forming optical system 3 included in the fluid analysis device 1 of the embodiment forms an image with the light applied to the blood flow X. In addition, the image forming optical system 3 adjusts the numerical aperture based on the speckle data acquired by the data acquiring unit 4 as described below. It is preferable that the speckle data is data of the speckle contrast, and at this point, the image forming optical system 3 adjusts the numerical aperture so that the speckle contrast becomes the maximum. Accordingly, the detection accuracy of the flow of the blood flow X can be enhanced. In addition, the details of the speckle contrast are described below.

The image forming optical system 3 mainly includes a first lens 31 that functions as a condensing lens that focuses the light 1 applied to the blood flow X, a diaphragm 32 that can change the opening diameter in a plane direction parallel to a Fourier surface, and a second lens 33 that functions as an imaging lens focusing on the imaging device 41 described below. The image forming optical system 3 can adjust the numerical aperture by adjusting the size of the diaphragm 32.

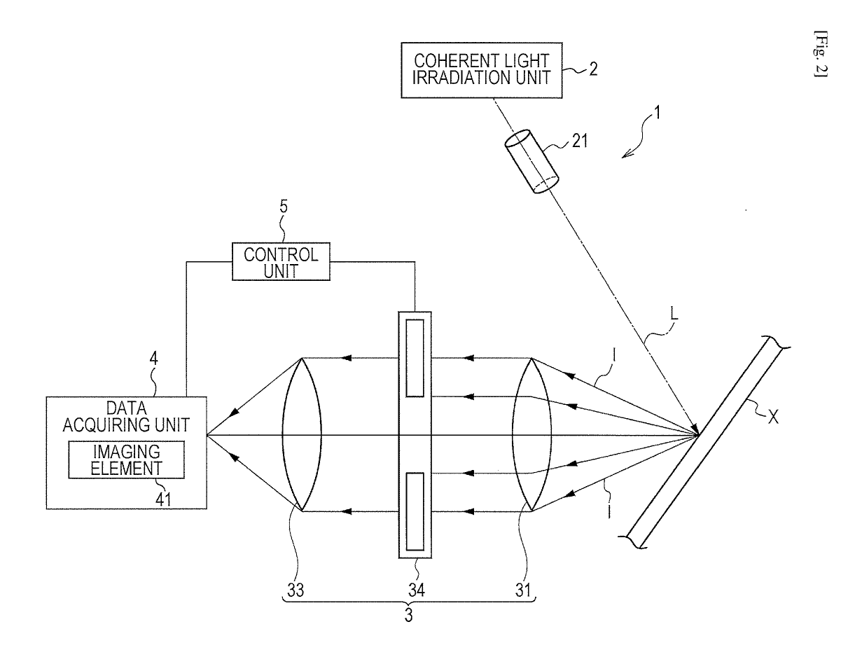

FIG. 2 is a diagram illustrating a concept of another device configuration of the fluid analysis device 1. As illustrated in FIG. 2, the image forming optical system 3 may have an optional spatial optical modulator 34, instead of the diaphragm 32. In the fluid analysis device 1 of the embodiment, the speckle contrast of the speckle image can be caused to be the maximum by controlling the opening of the spatial optical modulator 34, instead of adjusting the size of the diaphragm 32.

In addition, the fluid analysis device 1 of the embodiment can be operated by using a digital micro device (DMD). Further, at the position of the diaphragm 32 described above, an optional light valve that can electronically or mechanically control the numerical aperture can be provided. As a device that causes the image forming optical system 3 to change the numerical aperture, an optional optical modulating element such as a mechanical iris diaphragm, a liquid crystal shutter, an electrochromic device, and an electrophoretic device can be used.

<Data Acquiring Unit 4>

The data acquiring unit 4 included in the fluid analysis device 1 of the embodiment acquires the speckle data of the blood flow X irradiated with the coherent light L. Particularly, the data acquiring unit 4 can include the imaging device 41 as a CCD camera, and it is possible to capture the speckle image of the blood flow X with the imaging device 41 and to acquire the speckle data.

The data acquiring unit 4 can transmit a signal relating to the acquired speckle data to a control unit 5 described below, and adjusts the numerical aperture of the image forming optical system 3 by the control unit 5. Also, in the fluid analysis device 1 of the embodiment, in order to statistically process a speckle pattern described below and to accurately obtain data of the speckle contrast with respect to the numerical aperture, a pixel size of the image captured by the imaging device 41 is preferably smaller than a speckle grain size.

<Control Unit 5>

The fluid analysis device 1 of the embodiment can further include the control unit 5. In the control unit 5, a central processing unit (CPU), a memory, an input and output interface unit, a hard disk or the like may be provided. Details thereof are described in the fluid analysis method described below. However, the control unit 5 can generate the speckle data for each numerical aperture based on the speckle data acquired by the data acquiring unit 4. Specifically, the control unit 5 can generate data of the speckle contrast for each numerical aperture. Also, the control unit 5 can adjust the numerical aperture of the diaphragm 32 in the image forming optical system 3 based on the speckle data acquired by the data acquiring unit 4.

<Fluid Analysis Method>

Next, an example of the fluid analysis method of the blood flow X performed by the fluid analysis device 1 of the embodiment is described. The fluid analysis method includes a data acquiring step of irradiating the blood flow X with coherent light and acquiring speckle data of the fluid irradiated with the coherent light and a step of adjusting the numerical aperture of the image forming optical system 3 for forming an image with the light applied to the fluid based on the speckle data.

In the fluid analysis device 1 of the embodiment, the numerical aperture of the diaphragm 32 of the image forming optical system 3 is adjusted to an optimum value by the control unit 5 so that the movement of the scattered fluid of the blood flow X can be most distinctly observed from the speckle data acquired by the data acquiring unit 4. Particularly, the image forming optical system 3 adjusts the numerical aperture so that the speckle contrast becomes the maximum.

<Speckle Data>

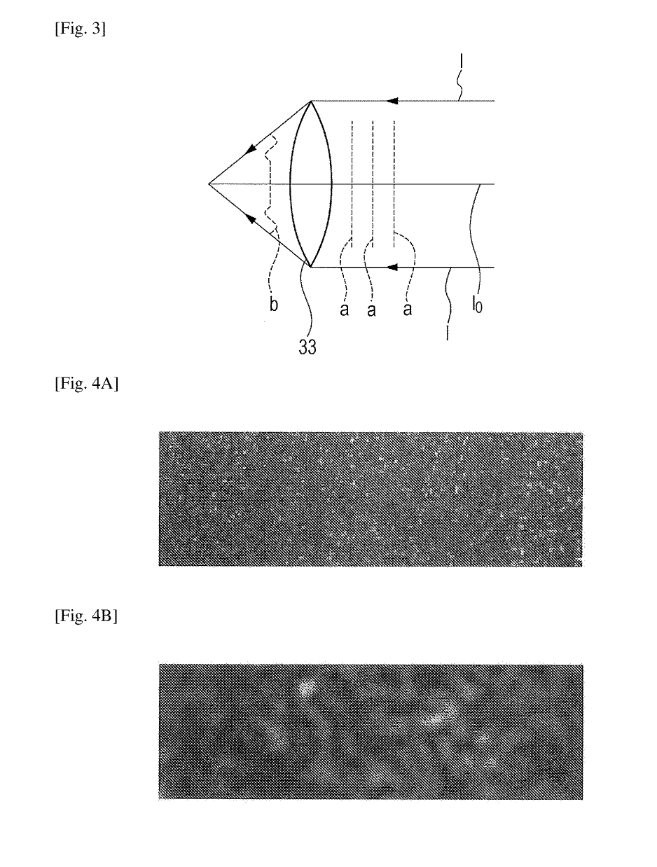

Here, the speckle data for adjusting the numerical aperture is described more specifically. FIG. 3 is a diagram illustrating a state of forming an image with light using the image forming optical system 3. As illustrated in FIG. 3, rays near an optical axis I₀ of the light applied to the blood flow X are not disturbed on the wave surface, but the rays are disturbed on the wave surface as it gets farther from the optical axis (in the drawing, see references a and b), and a speckle phenomenon occurs due to the random interference of the scattered wave. Accordingly, the imaging device 41 can capture a spot pattern image (speckle image) of the blood flow X.

At this point, the image forming optical system 3 causes the numerical aperture to be great so that angles and directions of the incident light and phases vary and the scattered waves are equalized. Therefore, the speckle contrast decreases. Meanwhile, when the image forming optical system 3 causes the numerical aperture to be small, the speckle contrast becomes great. Further, if the wave front aberration in the image forming optical system 3 becomes small, the main phase difference occurs due to the scatter from a rough surface of the blood flow X. At this point, if the phase difference applied by the scatter from the blood flow X is in a range of 1 cycle (−pi to pi), a phenomenon called “undeveloped speckle” by the image forming optical system 3 is generated so that a speckle pattern changes and the speckle contrast decreases on the imaging surface.

If the blood flow X is detected by using the change of the speckle data, it is considered that when the speckle contrast is great, the flow measurement sensitivity or the accuracy becomes high. Therefore, if the spatial resolution is negligible, the flow can be measured with high accuracy by reducing the numerical aperture of the image forming optical system 3 to be as small as possible in a range in which the speckle is not undeveloped.

The numerical aperture at this point varies in response to the phase difference occurring due to the blood flow X which is a measuring object, the aberration of the image forming optical system 3, the numerical aperture of the illumination system, and the sensitivity of the two-dimensional imaging device 41. The image forming optical system 3 changes the numerical aperture based on the speckle data (intensity of speckle contrast, speckle pattern, or the like) acquired by capturing the speckle image by the data acquiring unit 4.

As described above, as a device for adjusting the numerical aperture of the image forming optical system 3 based on the signal from the speckle data obtained from the control unit 5, an optional optical modulating element such as a mechanical iris diaphragm, a liquid crystal shutter, an electrochromic device, and an electrophoretic device can be used.

Subsequently, with reference to FIGS. 4A and 4B, the aforementioned undeveloped speckle and the sufficiently developed speckle are described below. FIGS. 4A and 4B are diagrams illustrating an example of the speckle image.

<Sufficiently Developed Speckle>

In general, a complex amplitude A at an optional observation point x on an image surface is given by Expression (1) below (see Speckle phenomena in optics: theory and applications, Chapter 2, pp. 7-23, Roberts & Company, Englewood, Colo., written by J. W. Goodman).

[Math.1]

A(x)=|A(x)|exp[iθ(x)] Expression (1)

In addition, in Expression (1), theta represents a phase.

A complex amplitude of the light made on an image surface by n points of a dispersed object such as the blood flow X is expressed by Expression (2) below.

$$\begin{matrix}[ Math . 2 ] & \\ A ( x ) = {\sum }_{k = 1}^{N} \frac{1}{\sqrt{N}} {a}_{k} ( x ) = \frac{1}{\sqrt{N}} {\sum }_{k = 1}^{N} \vert {a}_{k} ( x ) \vert \cdot exp ( i {φ}_{k} ) & Expression ( 2 )\end{matrix}$$

If the phase distribution of each light from N scatter points of the scattered object independently is the same, the probability density function with respect to a real portion Ar and an imaginary portion Ai of the complex amplitude is expressed by Expression (3) from the central limit theorem.

$$\begin{matrix}[ Math . 3 ] & \\ {P}_{r , i} ( {A}_{r} , {A}_{i} ) = \frac{1}{2 {πσ}^{2}} exp ( - \frac{{A}_{r}^{2} + {A}_{i}^{2}}{2 {σ}^{2}} ) & Expression ( 3 )\end{matrix}$$

Further, in Expression (3), sigma represents a standard deviation between Ar and Ai.

At this point, intensity I and a phase theta of the speckle are expressed by Expressions (4) and (5) below, respectively. Therefore, an intensity probability density function P_{N}(I) and a phase probability density function Pₜₕₑₜₐ(theta) are expressed by Expressions (6) and (7) below, respectively.

[Math.4]

I=Aᵣ²+Aᵢ² Expression (4)

$$\begin{matrix}[ Math . 5 ] & \\ θ = {tan}^{- 1} ( \frac{{A}_{i}}{{A}_{r}} ) & Expression ( 5 ) \\ [ Math . 6 ] & \\ {P}_{I} ( I ) = \frac{1}{2 {σ}^{2}} exp ( - \frac{I}{2 {σ}^{2}} ) & Expression ( 6 ) \\ [ Math . 7 ] & \\ {P}_{θ} ( θ ) = \frac{1}{2 π} & Expression ( 7 )\end{matrix}$$

The speckle expressed by the probability density functions can be called a sufficiently developed speckle, and a pattern illustrated in FIG. 4A is an example thereof.

<Undeveloped Speckle>

Meanwhile, if the phase is in a range of −pi<=theta<=pi, and the distribution is not even, the probability density function is expressed by a general Gauss probability density function of Expression (8) below.

$$\begin{matrix} [ Math . 8 ] & \\ {P}_{r , i} ( {A}_{r} , {A}_{i} ) = \frac{1}{2 {πσ}_{r} {{σ}_{i} ( 1 - {ρ}^{2} )}^{1 / 2}} \cdot exp [ - \frac{1}{{( 1 - ρ )}^{2}} \cdot ( \frac{Δ {A}_{r}^{2}}{{σ}_{r}^{2}} - \frac{2 ρ \cdot Δ {A}_{r} \cdot Δ {A}_{i}}{{σ}_{r} \cdot {σ}_{i}} + \frac{Δ {A}_{i}^{2}}{{σ}_{i}^{2}} ) ] & Expression ( 8 )\end{matrix}$$

Further, in Expression (8), rho is a correlation coefficient between Ar and Ai, delta Ar=Ar−<Ar>, and delta Ai=Ai−<Ai>.

The speckle expressed by the probability density function is called an undeveloped speckle, and a pattern illustrated in FIG. 4B is an example.

<Speckle Contrast>

The control unit 5 can convert information of the blood flow X into the speckle contrast by performing data processing on the speckle data acquired by the data acquiring unit 4. The speckle contrast (CS) is expressed by Expression (9) below. In addition, the velocity of the light scattered fluid such as the blood flow X is inversely proportional to a square of the speckle contrast. (See Opt. Commun. 37 (5), p. 325 (1981) or the like)

$$\begin{matrix}[ Math . 9 ] & \\ {C}_{S} = \frac{σ}{〈 I 〉} & Expression ( 9 )\end{matrix}$$

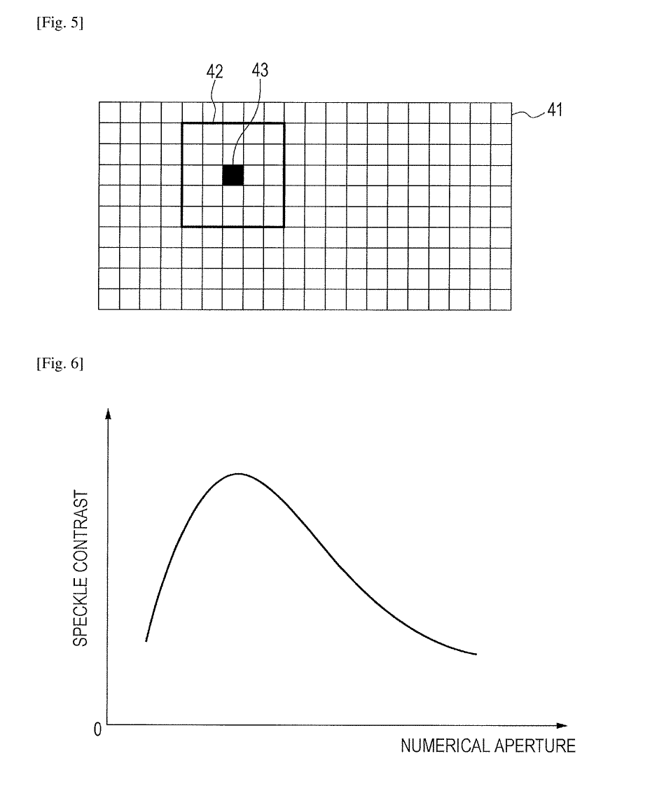

FIG. 5 is a diagram schematically illustrating a state of mapping the speckle image. With respect to the speckle image illustrated in FIGS. 4A and 4B, the entire range of an image 41 for each pixel is mapped as illustrated in FIG. 5 so that the image of the blood flow X can be generated, and the movement and the flow of the blood flow X can be observed.

As illustrated in FIG. 5, for example, an area of the image 41 in which statistical processing for calculating the speckle contrast is performed is set to be a square area 42 in which five pixels are arranged respectively in horizontal and vertical directions. For example, the speckle contrast in a center 43 of the square area 42 can be calculated as the speckle contrast of the square area. An optional range can be set as the statistical processing area depending on the resolution of the speckle image and the measurement accuracy of the flow of the blood flow X.

FIG. 6 is a graph illustrating a relationship between the numerical aperture and the speckle contrast. In the graph illustrated in FIG. 6, the image forming optical system 3 can adjust the numerical aperture so that the speckle contrast becomes the maximum. Accordingly, the detection accuracy of the flow of the blood flow X can be enhanced.

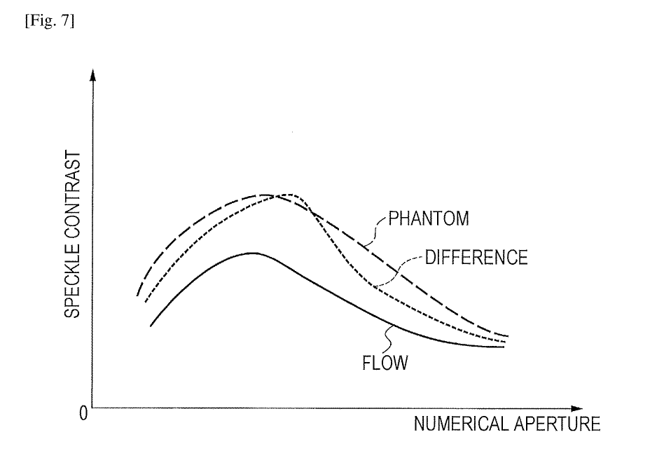

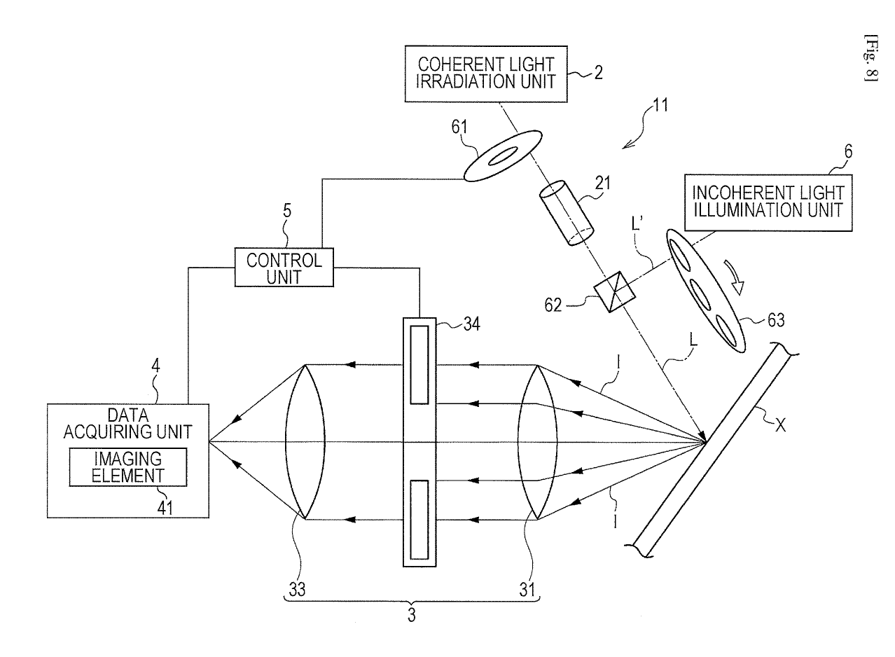

In addition, the data acquiring unit 4 acquires data of speckle contrast other than that of the fluid such as the blood flow X, and the image forming optical system 3 can adjust the numerical aperture so that the speckle contrast of the blood flow X and the speckle contrast other than that of the blood flow X becomes the maximum. FIG. 7 is a graph illustrating a relationship between the numerical aperture and the speckle contrast. Particularly, FIG. 7 is a graph illustrating the numerical aperture and the difference between the speckle contrast of the blood flow X and the speckle contrast other than that of the blood flow X. The detection accuracy of the flow of the blood flow X can be further increased by adjusting the numerical aperture so that the difference (difference in FIG. 7) between the speckle contrast of the blood flow X (flow in FIG. 7) and the speckle contrast other than that of the blood flow X (phantom in FIG. 7) becomes the maximum.

In the above, the method of adjusting the numerical aperture so that the speckle contrast becomes the maximum is described, but the image forming optical system 3 may adjust the numerical aperture based on the granularity or the stripe density of the speckle image. With reference to FIGS. 4A and 4B, the speckle image illustrated in FIG. 4A has granularity greater than that of the speckle image illustrated in FIG. 4B. Meanwhile, the speckle image illustrated in FIG. 4B has stripe density greater than that of the speckle image illustrated in FIG. 4A. The control unit 5 can control the numerical aperture in response to the value of the granularity and stripe density thereof, by distributing, for example, how much pixels having luminance values which are the same or in a certain range are continued and adjacent to each other. In this manner, the image forming optical system 3 can adjust the numerical aperture so that the granularity of the speckle image becomes greater.

As described above, since in the fluid analysis device 1 of the embodiment, the image forming optical system 3 adjusts the numerical aperture based on the speckle data, it is possible to enhance the detection accuracy of the flow of the blood flow X. Particularly, the blood flow X can be accurately observed by causing the image forming optical system 3 to adjust the numerical aperture so that the speckle contrast becomes the maximum by using the data of the speckle contrast as the speckle data. In addition, the blood flow X can be accurately observed by causing the image forming optical system 3 to adjust the numerical aperture so that the difference between the speckle contrast of the blood flow X and the speckle contrast other than that of the blood flow X becomes the maximum.

### Modification Example

FIG. 8 is a diagram schematically illustrating a configuration example of the fluid analysis device 11 of the modification example of the embodiment. The fluid analysis device 11 of the modification example of the embodiment is different from the fluid analysis device 1 of the first embodiment described above in that the fluid analysis device 11 includes an incoherent light irradiation unit 6 that irradiates the blood flow X with incoherent light L′. Therefore, herein, the configuration of the incoherent light irradiation unit 6 and the function thereof are mainly described.

As illustrated in FIG. 8, when a bright field image or a fluorescence image is observed by irradiating the blood flow X with the incoherent light L by the incoherent light irradiation unit 6, and using the image forming optical system 3 which is the same as the coherent light irradiation unit 6, a necessary resolution and brightness are different. Therefore, the image forming optical system 3 can adjust the diaphragm 32 so that the size of the aperture becomes optimal for the speckle image and the bright field and fluorescence image. As the incoherent light irradiation unit 6 is not particularly limited, a visible light laser with low coherency such as a Xe lamp can be used.

Further, in the fluid analysis device 11 of this modification example, a rotating chopper 61 may be provided between the coherent light irradiation unit 2 and the beam expander 21. In addition, a polarization beam splitter 62 may be provided between the beam expander 21 and the blood flow X. For example, the parallel light from the Xe lamp can pass through a rotating band pass filter 63 that can block the red light, the green light, and the blue light at an even time interval, and be incident to and reflected on the polarization beam splitter 62. The rotating chopper 61 and the rotating band pass filter 63 can be synchronized, and the same position of the blood flow X can be illuminated with red, green, and blue naturally emitted light and a near infrared laser beam at a specific time interval.

In addition, the configurations and the effects of the fluid analysis system of the embodiment in addition to the above are the same as in those in the first embodiment.

### 2. Second Embodiment

Subsequently, the fluid analysis system according to the second embodiment of the disclosure is described. In the fluid analysis system of the second embodiment, the control unit 5 can be provided in a different device from the fluid analysis devices 1, and 11, compared with the fluid analysis device 1 of the first embodiment and the fluid analysis device 11 of the modification example of the first embodiment. Accordingly, for example, the control unit 5 can adjust the numerical aperture based on the speckle data in the image forming optical system 3 of the fluid analysis device 1 via a network.

The network includes, for example, a public network such as the Internet, a telephone network, a satellite communication network, and a broadcast communication path, or a leased line network such as a wide area network (WAN), a local area network (LAN), an internet protocol-virtual private network (IP-VPN), an Ethernet (registered trade mark), and a wireless LAN, regardless of being wired or wireless. In addition, the network may be a communication channel network exclusively provided in the fluid analysis system of the embodiment.

Also, a server, an image display apparatus, or the like may be provided in the fluid analysis system of the embodiment. In this case, the fluid analysis devices 1, and 11, a server, and an image display apparatus may be directly connected, or may be connected in a communicable manner via a network.

In addition, the configurations and the effects of the fluid analysis system of the embodiment in addition to the above are the same as in those in the first embodiment.

FIG. 13 shows a diagram of a medical imaging system, specifically, an endoscopy system 130, according to some embodiments. As shown in FIG. 13, the endoscopy system 130 has a light source 132. Light source 132 may include a coherent light irradiation unit 2, an incoherent light illumination unit 6, or both a coherent light irradiation unit 2, an incoherent light illumination unit 6, as discussed above. The light source 132 emits light that is provided to an endoscope body 134 to perform endoscopy.

The endoscope body 134 may include a lighting optical system 136 to receive the light from light source 132. The lighting optical system 136 may include any suitable optical components, such as one or more lenses, one or more optical fibers, a beam expander 21, a beam splitter such as polarization beam splitter 62, a filter such as rotating band pass filter 63, or any suitable combination of such components. However, the techniques described herein are not limited to the lighting optical system 136 being included within the endoscope body 134, as in some embodiments on or more components of the lighting optical system 136 may be provided external to the endoscope body 134. Light may be emitted from the lighting optical system to 136 to illuminate a region of an organism, such as the human body, to perform endoscopy.

The endoscope body 134 may also include an image forming optical system 3 and a data acquiring unit 4 which may include an imaging element 41, as described above.

The endoscopy system 130 may include a control unit 5 that receives data from the data acquiring unit 4 and controls the image forming optical system 3. The control unit 5 and/or the data acquiring unit 4 may include a processor that processes the received image data to obtain speckle data, such as a speckle contrast. In some embodiments, the control unit 5 may control the light source 132.

The endoscopy system 130 may include a display 138 for display of an image produced by the data acquiring unit 4 and/or the control unit 5. Display 138 may be any suitable type of display for displaying images to a user.

In some embodiments, a medical imaging system may include a microscope including an optical apparatus as described above and shown in FIG. 1, by way of example.

Also, the disclosure may have configurations as described below.

(1)

A fluid analysis device including a coherent light irradiation unit that irradiates fluid with coherent light;

an image forming optical system that forms an image with the light applied to the fluid; and

a data acquiring unit that acquires speckle data of the fluid, in which the image forming optical system adjusts a numerical aperture based on the speckle data.

(2)

The fluid analysis device according to (1), in which the speckle data is data of speckle contrast.

(3)

The fluid analysis device according to Item (2), in which the image forming optical system adjusts the numerical aperture so that the speckle contrast becomes the maximum.

(4)

The fluid analysis device according to (2) or (3), in which the data acquiring unit acquires data of the speckle contrast other than that of the fluid, and the image forming optical system adjusts the numerical aperture so that a difference between speckle contrast of the fluid and speckle contrast other than that of the fluid becomes the maximum.

(5)

The fluid analysis device according to any one of (1) to (4), further including an incoherent light irradiation unit that irradiates the fluid with incoherent light.

(6)

The fluid analysis device according to any one of (1) to (5), in which the fluid is blood.

(7)

A fluid analysis method including:

irradiating fluid with coherent light, and acquiring speckle data of the fluid irradiated with the coherent light; and

adjusting a numerical aperture of a coupling optical system that forms an image with the light applied to the fluid based on the speckle data.

(8)

A program for causing a fluid analysis device to perform:

a data acquiring function of acquiring speckle data of a fluid irradiated with coherent light; and

a function of adjusting a numerical aperture of a coupling optical system that forms an image with the light applied to the fluid based on the speckle data.

(9)

A fluid analysis system including:

a coherent light irradiation unit that irradiates fluid with coherent light;

an image forming optical system that forms an image with the light applied to the fluid; and

a data acquiring unit that acquires speckle data of the fluid,

in which the image forming optical system adjusts a numerical aperture based on the speckle data.

Some embodiments relate to an imaging apparatus that includes: a light source that emits coherent light to image an object;

an optical apparatus that optically processes light from the object, the optical apparatus having a numerical aperture; an imager that receives the light optically processed by the optical apparatus; and circuitry configured to obtain speckle data based on the light received by the imager and control the numerical aperture of the optical apparatus based on the speckle data.

In some embodiments, the speckle data is a speckle contrast.

In some embodiments, the circuitry is configured to control the numerical aperture of the optical apparatus to increase the speckle contrast.

In some embodiments, the circuitry is configured to control the numerical aperture of the optical apparatus to maximize the speckle contrast.

In some embodiments, the object comprises a fluid.

In some embodiments, the circuitry is configured to produce image data based on the speckle data, the image data indicating a location of the fluid.

In some embodiments, the circuitry is configured to control the numerical aperture of the optical apparatus to increase a difference between speckle data of the fluid and speckle data of a non-fluid.

In some embodiments, the speckle data is a speckle contrast and the circuitry is configured to control the numerical aperture of the optical apparatus to maximize a difference between the speckle contrast of the fluid and speckle contrast of a non-fluid.

In some embodiments, the fluid comprises blood and the imaging apparatus images blood.

In some embodiments, the circuitry includes a controller.

In some embodiments, the imager has a pixel size smaller than a speckle grain size of the light from the object.

In some embodiments, the light source is a first light source and the imaging apparatus further includes: a second light source that emits incoherent light to image the object, wherein the imager images the object using the incoherent light.

In some embodiments, the circuitry is configured to control the numerical aperture of the optical apparatus by changing a size of an opening in the optical apparatus.

In some embodiments, the optical apparatus comprises a diaphragm.

In some embodiments, the optical apparatus comprises a spatial optical modulator.

Some embodiments relate to an imaging method, including: emitting coherent light to image an object; optically processing light from the object using an optical apparatus having a numerical aperture; obtaining speckle data based on the light optically processed by the optical apparatus; and

controlling the numerical aperture of the optical apparatus based on the speckle data.

Some embodiments relate to a medical imaging system, including:

an imaging apparatus, including: a light source that emits coherent light to image an object; an optical apparatus that optically processes light from the object, the optical apparatus having a numerical aperture; an imager that receives the light optically processed by the optical apparatus; and

circuitry configured to obtain speckle data based on the light received by the imager and control the numerical aperture of the optical apparatus based on the speckle data.

In some embodiments, the fluid is blood and the imaging apparatus images blood.

In some embodiments, the medical imaging system includes a microscope or an endoscope.

In some embodiments, the medical imaging system further includes a display that displays an image produced by the imaging apparatus.

### EXAMPLES

Effects of the disclosure are described in detail with reference to examples of the disclosure.

### Example 1

Fluid is observed using the fluid analysis device illustrated in FIG. 1. Specifically, an external resonance semiconductor laser (CW oscillation, wavelength: 780 nanometer, single vertical mode, line width: 300 kHz (1 ms), transverse mode: TEM00, output number: up to 100 mW) manufactured by Sacher Lasertechnik is used as a light source. Also, a laser beam emitted from the light source is expanded by a beam expander manufactured by Edmund Optics, and a phantom formed as a channel is irradiated with a parallel beam.

In a structure of the phantom, alumina particles as a light diffusion agent and red ink as a light absorbent are mixed in an ultraviolet curing resin, and an absorption coefficient and an equivalent scatter coefficient in a wavelength of 780 nanometer are set to be 0.07 per millimeter and 1.08 per millimeter so as to be the same as those of an inner wall of a stomach of a human.

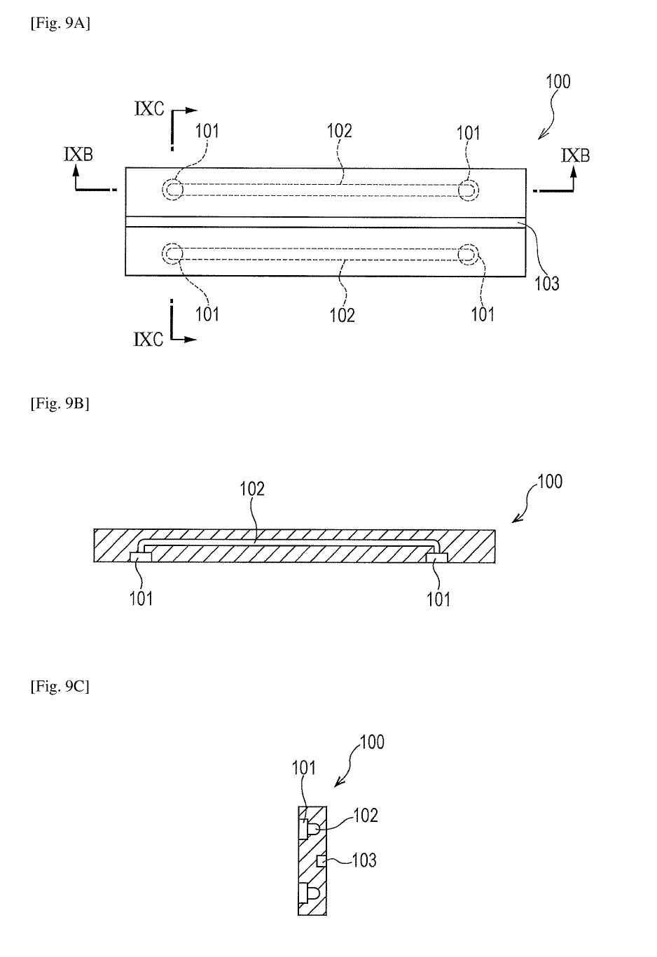

FIGS. 9A to 9C are diagrams illustrating a configuration of the tool 100 circulate the fluid. A cross-sectional view taken along line IXB-IXB in a front view of FIG. 9A is illustrated in FIG. 9B, and a cross-sectional view taken along line IXC-IXC is illustrated in FIG. 9C. Specifically, as illustrated in FIGS. 9A to 9C, an opening portion 101 and a channel 102 of which a cross section has a 1 millimeter angle are formed in positions having a depth of 200 micrometers from an observation surface, and a tube is connected to an injection and discharge port provided on a rear side with respect to the observation surface. In addition, artificial blood flows from the opening portion 101 to the channel 102 at a velocity of about 10 millimeter/sec with a syringe pump. In Example 1, drinkable milk is used as the artificial blood. A reference number 103 denotes a focusing member.

Specifically, two sheets of spherical planoconvex quartz lenses having a focal length of 150 millimeter and an opening diameter of 30 millimeter are used as the image forming optical system 3, the diaphragm 32 (iris diaphragm) is arranged on a Fourier surface therebetween, and a diameter of the aperture is adjustable in a range of 3 millimeter to 15 millimeter.

A focus is adjusted so that an imaging surface of a CCD camera is identical to an image surface of the data acquiring unit 4. An industrial CCD camera (XCD-V60) manufactured by Sony Corporation is used as an imaging CCD camera. In addition, the camera has a pixel size sufficiently smaller than a speckle grain size so that an intensity of a speckle pattern is a resolution sufficient for statistical processing (square of which one side is 7.4 micrometers).

A signal from the CCD camera is output as a bitmap file of 8 bits, VGA, 60 fps, and is obtained by a PC according to IEEE1394b, to perform statistical processing. An image of the flow is generated by setting an image area for performing statistical processing in order to calculate speckle contrast as a square area having five pixels respectively in horizontal and vertical directions, setting speckle contrast at this point as speckle contrast in the center of the area, and mapping the entire image.

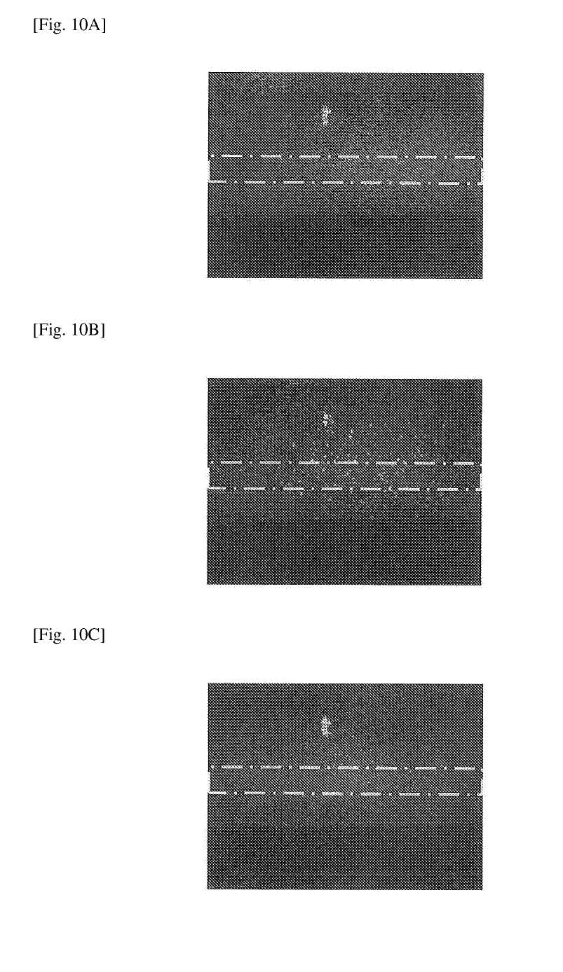

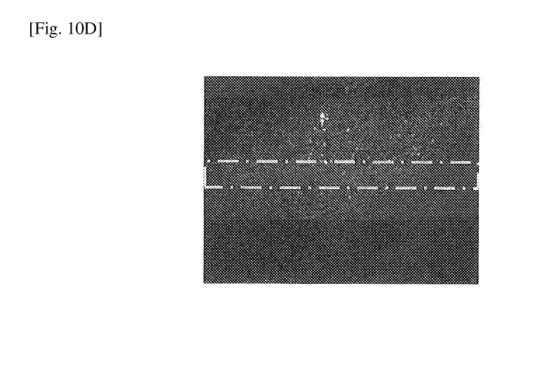

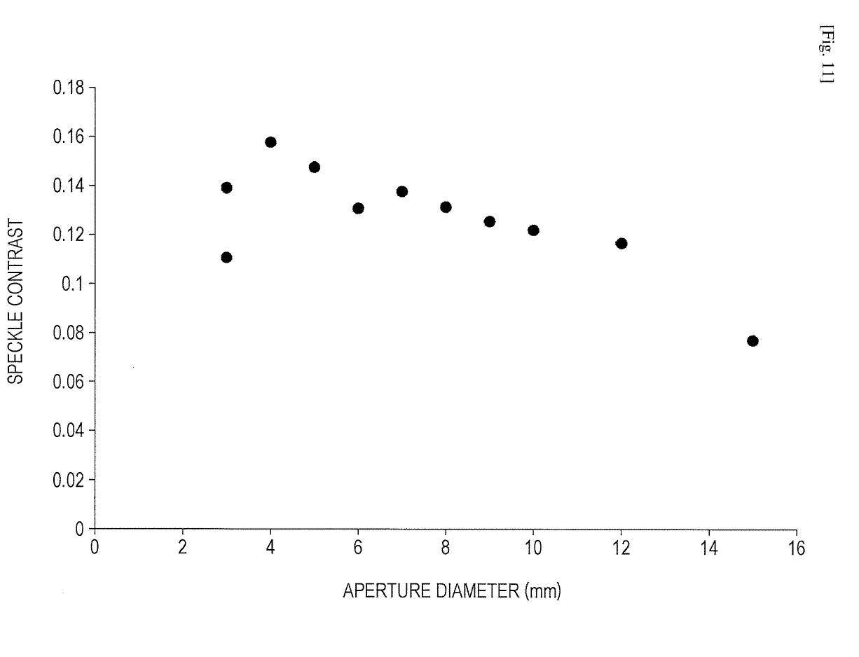

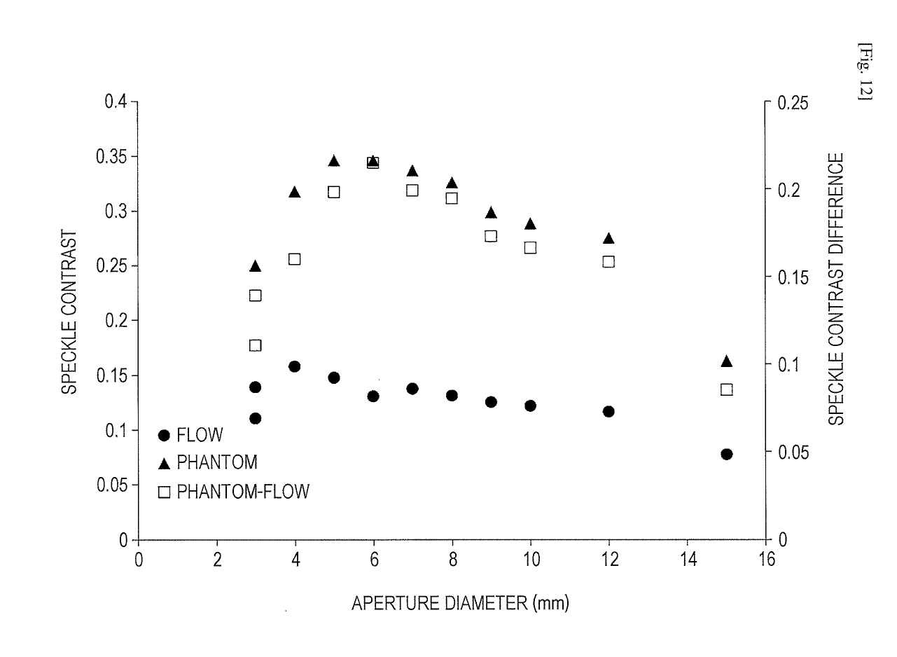

FIGS. 10A to 10D are diagrams illustrating an example of the speckle image obtained in this manner. As illustrated in FIGS. 10A to 10D, portions enclosed with frames of alternate long and short dashed lines in all patterns correspond to channels. FIGS. 10A and 10B illustrate speckle when the flow of the fluid X is stopped, and FIGS. 10C and 10D illustrate the fluid X flowing. Also, FIGS. 10A and 10C illustrate that the opening diameter of the iris diaphragm is 12 millimeter, and FIGS. 10B and 10D illustrate that the opening diameter is 4 millimeter. It can be observed that speckle patterns and contrast become different according to whether the fluid flows or not or according to the size of the aperture, from the image illustrated in FIGS. 10A to 10D.

From the image information, the speckle contrast is calculated in the method above with reference to Expression (9). Particularly, the speckle contrast for each size of the aperture can be obtained by obtaining a standard deviation sigma and an average <I> from the frequency distribution of the luminance gradation obtained by subtracting a value corresponding to a dark current for each pixel with respect to the area in which the illuminance near the center of the channel image is even.

FIG. 11 is a graph illustrating a relationship between the numerical aperture and the speckle contrast with respect to the speckle images illustrated in FIGS. 10A to 10D. As illustrated in FIG. 11, in the optical system and the sample according to Example 1, it is found that when the size of the aperture is 4 millimeter, the speckle contrast is obtained as a maximum value. That is, it is found that the sensitivity and the accuracy become the maximum by setting the numerical aperture of the image forming optical system 3 to be 4 millimeter in order to visualize the flow of the fluid. In this manner, the numerical aperture of the image forming optical system 3 can be controlled so that the speckle contrast becomes the maximum.

### Example 2

In Example 2, a test is performed in the same manner as in Example 1 except for controlling the aperture so that the difference between the speckle contrast and speckle contrast other than that of the fluid X becomes the maximum instead of controlling the aperture so that the speckle contrast becomes the maximum.

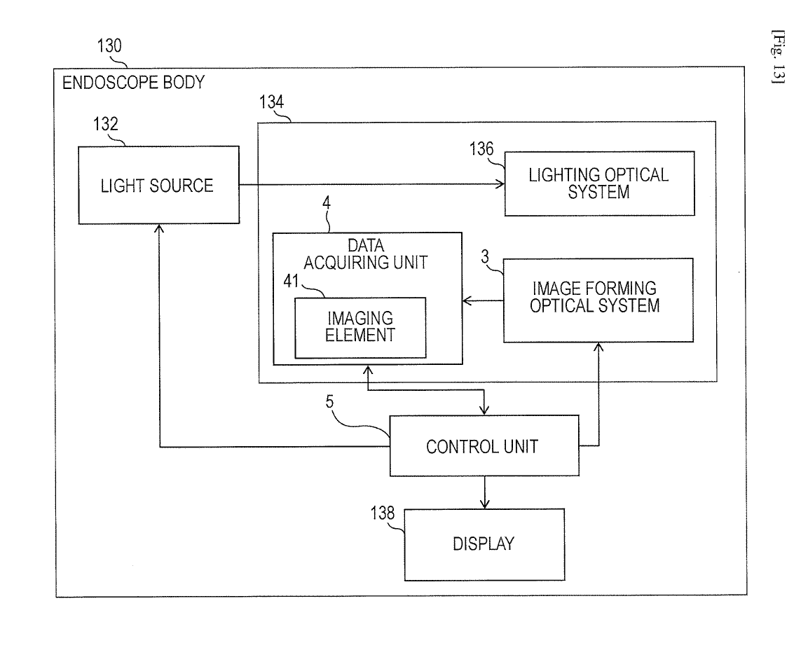

FIG. 12 is a graph illustrating a relationship between the numerical aperture and the difference between the speckle contrast and the speckle contrast other than that of the fluid X with respect to the speckle images illustrated in FIGS. 10A to 10D. As illustrated in FIG. 12, in Example 2, it is found that the sensitivity and the accuracy become the maximum by setting the size of the aperture of the image forming optical system 3 to be about 6 millimeter. In this manner, the numerical aperture of the image forming optical system 3 can be controlled.

### Example 3

A test is performed in the same manner as in Example 1 except for using the image forming optical system 3 illustrated in FIG. 2 instead of the image forming optical system 3 used in Examples 1 and 2. Specifically, an image of the flow is captured by controlling the aperture in real time by the liquid crystal transmission-type spatial optical modulator manufactured by Holoeye photonics installed in substitution for the iris diaphragm. In addition, the size of the aperture of which a speckle pattern changes from a particle shape to a stripe shape is obtained while the aperture of the spatial optical modulator is controlled, and the image of the flow with the numerical aperture and the shape thereof is captured. Accordingly, the test result which is the same as that in Example 1 can be obtained.

### Example 4

A test is performed in the same manner as in Examples 1, 2, and 3 except for using the fluid analysis device 11 illustrated in FIG. 8 instead of the fluid analysis device 1 used in Examples 1, 2, and 3. Particularly, based on an optical path of Example 1 or the like, the rotating chopper 61 is provided in front of the beam expander 21, and the broad band polarization beam splitter 62 which is valid when the wavelength is in the range of 400 nanometer to 800 nanometer is provided behind the beam expander 21. The polarization beam splitter 62 is arranged so as to be penetrated by a laser in the wavelength of 780 nanometer. Also, the parallel light from the Xe lamp can pass through the rotating band pass filter 63 in which the blockage of red, green, and blue changes at an even time interval, and be incident to and reflected on the polarization beam splitter 62. The rotating chopper 61 and the rotating band pass filter 63 are synchronized, and the same position on the sample surface can be illuminated with red, green, and blue naturally emitted light and a near infrared laser beam at the same time interval. The rotating chopper 61 and the rotating band pass filter 63 are synchronized with an imaging camera (the imaging device 41), the images illuminated with the red, green, and blue light and a near infrared laser beam are repeatedly obtained, and the image data is sent to a PC.

In the diaphragm on a Fourier surface, the spatial optical modulator 34 which is the same as in Example 3 is provided. When the spatial resolution is necessary for bright field observation, the numerical aperture is set to be great, and when the sensitivity and the accuracy of the flow in the speckle image are necessary, the numerical aperture is set so that the sensitivity and the accuracy of the flow detection in the method of Example 2 become high, and this aperture control is repeated. Accordingly, a good bright field image and a good image of a flow in the same observation position can be simultaneously observed in real time. In addition, in Example 4, the Xe lamp is used as the incoherent light irradiation unit 6, but a visible light laser with sufficiently low coherency may be used.

In Example 4, it is found that optimum sensitivity, accuracy, resolution, and a focal depth are obtained when the image forming optical system is used together with the bright field image, the fluorescence image, and the like.

In the above, in Examples 1 to 4, it can be found that the blood flow is accurately observed by adjusting the numerical aperture of the image forming optical system in the fluid analysis device of the disclosure.

It should be understood by those skilled in the art that various modifications, combinations, sub-combinations and alterations may occur depending on design requirements and other factors insofar as they are within the scope of the appended claims or the equivalents thereof.

### REFERENCE SIGNS LIST

1, 11 Fluid analysis device
2 Coherent light irradiation unit
3 Image forming optical system
4 Data acquiring unit
5 Control unit
6 Incoherent light irradiation unit
31 First lens
32 Diaphragm
33 Second lens
X Fluid (blood flow)

1. An imaging apparatus, comprising:
a light source that emits coherent light to image an object;
an optical apparatus that optically processes light from the object, the optical apparatus having a numerical aperture;
an imager that receives the light optically processed by the optical apparatus; and
circuitry configured to obtain speckle data based on the light received by the imager and control the numerical aperture of the optical apparatus based on the speckle data.

2. The imaging apparatus of claim 1, wherein the speckle data is a speckle contrast.

3. The imaging apparatus of claim 2, wherein the circuitry is configured to control the numerical aperture of the optical apparatus to increase the speckle contrast.

4. The imaging apparatus of claim 3, wherein the circuitry is configured to control the numerical aperture of the optical apparatus to maximize the speckle contrast.

5. The imaging apparatus of claim 1, wherein the object comprises a fluid.

6. The imaging apparatus of claim 5, wherein the circuitry is configured to produce image data based on the speckle data, the image data indicating a location of the fluid.

7. The imaging apparatus of claim 5, wherein the circuitry is configured to control the numerical aperture of the optical apparatus to increase a difference between speckle data of the fluid and speckle data of a non-fluid.

8. The imaging apparatus of claim 7, wherein the speckle data is a speckle contrast and the circuitry is configured to control the numerical aperture of the optical apparatus to maximize a difference between the speckle contrast of the fluid and speckle contrast of a non-fluid.

9. The imaging apparatus of claim 5, wherein the fluid comprises blood and the imaging apparatus images blood.

10. The imaging apparatus of claim 1, wherein the circuitry comprises a controller.

11. The imaging apparatus of claim 1, wherein the imager has a pixel size smaller than a speckle grain size of the light from the object.

12. The imaging apparatus of claim 1, wherein the light source is a first light source and the imaging apparatus further comprises:
a second light source that emits incoherent light to image the object,
wherein the imager images the object using the incoherent light.

13. The imaging apparatus of claim 1, wherein the circuitry is configured to control the numerical aperture of the optical apparatus by changing a size of an opening in the optical apparatus.

14. The imaging apparatus of claim 13, wherein the optical apparatus comprises a diaphragm.

15. The imaging apparatus of claim 13, wherein the optical apparatus comprises a spatial optical modulator.

16. An imaging method, comprising:
emitting coherent light to image an object;
optically processing light from the object using an optical apparatus having a numerical aperture;
obtaining speckle data based on the light optically processed by the optical apparatus; and
controlling the numerical aperture of the optical apparatus based on the speckle data.

17. A medical imaging system, comprising:
an imaging apparatus, comprising:
a light source that emits coherent light to image an object;
an optical apparatus that optically processes light from the object, the optical apparatus having a numerical aperture;
an imager that receives the light optically processed by the optical apparatus; and
circuitry configured to obtain speckle data based on the light received by the imager and control the numerical aperture of the optical apparatus based on the speckle data.

18. The medical imaging system of claim 17, wherein the fluid is blood and the imaging apparatus images blood.

19. The medical imaging system of claim 17, wherein the medical imaging system comprises a microscope or an endoscope.

20. The medical imaging system of claim 17, further comprising a display that displays an image produced by the imaging apparatus.
